(19) United States
(12) Patent Application Publication
(10) Pub. No.: US 2022/0155274 A1
(43) Pub. Date: May 19, 2022

(54) SENSORS FOR THE EVALUATION OF THE QUALITY OF MEAT FOOD PRODUCTS

(71) Applicant: SAFER SMARTLABELS S.R.L., Pavia (IT)

(72) Inventors: Raffaela BIESUZ, Torre d'Isola (Pavia) (IT); Paolo QUADRELLI, Pavia (IT); Lisa Rita MAGNAGHI, Torre d'Isola (Pavia) (IT)

(21) Appl. No.: 17/599,196

(22) PCT Filed: Mar. 30, 2020
(86) PCT No.: PCT/IB2020/052998

§ 371 Date: Sep. 28, 2021

(30) Foreign Application Priority Data
Mar. 29, 2019 (IT) 102019000004741

(51) Int. Cl. G01N 33/12; G01N 21/80

(52) U.S. Cl. CPC G01N 33/12; G01N 21/80

(57) ABSTRACT

A system of sensors sensitive to a feature indicative of freshness of a food represented by meat is provided. The system of sensors includes a sensor having an indicator sensitive to pH change and a sensor having a binder sensitive to thiols or sulfur-containing compounds concentration.

### TECHNICAL FIELD OF THE INVENTION

The present invention applies to the field of packaging fresh food products and, in particular, meat.

### BACKGROUND

Meat is one of the main food products, for its high content of easily assimilable proteins, high caloric value fats, vitamins and trace elements necessary for the correct functioning of metabolic processes. In the last decades, a continuous increase of meat consumption was observed and, in parallel, the need, for the industries involved in production and processing of meat, to always find new solutions for classification and evaluation of degradation processes, has increased, this product being, especially for some types, a highly perishable fresh food (see chicken and fish).

Mostly, in the past decades, an awareness of the final consumer has grown, increasingly attentive to freshness and quality of this and other products related to human nutrition.

Therefore, on one side, the need is felt of having a continuous monitoring, a fast screening, based on simple, economic, highly efficient and effective methods for evaluating the quality and the freshness of the meat on a large scale; on the other side, we have the consumer, who today requires to be able to autonomously check, in a clear way, on the supermarket shelf, the status of the product he is purchasing. Smart labels, fitted directly in the package, with one or more sensors adapted to monitor the status of the product without the need of a third-party intervention, are designed with this in mind.

The colorimetric sensors, based on indicators able to change color subsequent to the reaction with one or more volatile compounds developing with the degradation progress directly on the meat packages are “in theory” the winning monitoring method. They are simple, practical and economic devices; furthermore, they do not require instrumentation. The color changes can be measured with a simple camera, but often a naked eye analysis is possible, using transparent films to close the package.

The pH indicators are the more suitable for the detection of microbial metabolites, since these molecules are mostly weak acids or bases.

In the numerous studies found in literature on the preparation of colorimetric sensors, attention is always directed to identification, if not sometimes determination, of biogenic amines. They are undoubtedly highly toxic compounds, and if introduced into the body can lead to serious consequences.

The most known cases of food poisoning are due to the ingestion of great amounts of histamine, especially coming from poorly preserved cheese or fish; at the same time, secondary amines, as cadaverine and putrescine, may react with nitrite to form heterocyclic nitrosamines, recognized as carcinogenic.

The main goal is safeguard consumer's health and it is important to have a clear signal that indicates that the product should not be consumed, even if it were within the maximum recommended date for consumption. This, although unlikely, could still happen if the cold chain had not been maintained or if the product had been accidentally contaminated, due to high perishability.

However, normally, the production of amines, in any type of meat, takes place only in a second and last decomposition phase, and generally it happens temporally beyond the terms within which its consumption is recommended. The smell and appearance (the color change), the slimy appearance for chicken meat, give, beyond any response of a sensor, which still helps, clear indications to abstain from the consumption.

Instead, a period that elapses between the purchase of meat from the supermarket shelf and actual consumption exists, even within the recommended expiration, or close thereto, in which doubts legitimately arise about the meat status. Understanding where one is arrived and maybe deciding for a food treatment rather than another, could be extremely useful and very well seen by the consumer.

However, there are no systems available to provide indications about the meat preservation status in this period of time.

### SUMMARY OF THE INVENTION

The authors of the present invention surprisingly found a system that allows to control the entire decomposition process of the meat and other fresh foods, even in its early phases, directly by the consumer.

### OBJECT OF THE INVENTION

A first object of the present invention is represented by a system of sensors (array) sensitive to a feature indicative of the freshness of a food product.

In a particular aspect, such food product is meat. In a second object, the invention describes the process for preparing such sensor.

A container or a container element for food, comprising a sensor or a system of sensors sensitive to one or more features indicative of the freshness of a fresh food product, represents a further object of the present invention.

According to a further object, a litmus paper is described that uses a sensor according to the present invention.

### BRIEF DESCRIPTION OF THE FIGURES

FIGS. 1A-F: structure and features of some indicators according to the present invention.

FIG. 2: an example of a system of three indicators (A,B,C) according to the present invention.

FIG. 3: Array of sensors for real sample analysis

FIG. 4: Comparison between the colors of the sensors during degradation in refrigerator—chicken meat.

FIG. 5: a) Descriptive model of the degradation process with identification of SAFE, WARNING and HAZARD areas; b) Validation by projection of external samples.

FIG. 6: A) Results of gas chromatographic analysis carried out at Centro Grandi Strumenti, CGS, University of Pavia, Italy, from air drawn in the head space of samples belonging to each of the three degradation phases, as attributed based on the color of the device. Duplicate Analysis B) Result of HPLC-ESI/MS analysis carried out at Centro Grandi Strumenti, CGS, University of Pavia, Italy, on three samples of chicken meat at same degradation degree as those on which the analysis reported in FIG. 6A is carried out. Duplicate Analysis.

FIG. 7: a) Adsorption kinetics of the binder in solid phase; b) Color reproducibility.

FIG. 8: a) Optimization of initial pH; b) Blank analysis in air.

FIG. 9: Comparison between sensor colors during degradation at room temperature, in case of beef.

FIG. 10: Descriptive model of the degradation process with identification of SAFE, WARNING and HAZARD areas relating to beef.

FIG. 11: Comparison between sensor colors during degradation at room temperature, in case of pork.

FIG. 12: Descriptive model of the degradation process with identification of SAFE, WARNING and HAZARD areas relating to pork.

FIG. 13: Comparison between the sensor colors during the degradation at room temperature, in the case of cod samples.

FIG. 14: Descriptive model of the degradation process with identification of SAFE and HAZARD areas relating to cod samples.

FIG. 15: Array of sensors for real sample analysis in the polymer version with derivatized EvOH.

FIG. 16: Comparison between sensor colors during degradation at room T, in case of chicken meat.

FIG. 17: a) Descriptive model of the degradation process in case of chicken meat with identification of SAFE, WARNING and HAZARD areas; b) Validation by projection of external samples.

FIG. 18: Example label with the color legend for reading the sensor.

FIG. 19: Array of sensors for real sample analysis in case of fish meat.

FIG. 20: Comparison between sensor colors during degradation at room T, in case of fish meat.

FIG. 21: a) Descriptive model of the degradation process in case of fish meat with identification of SAFE and HAZARD areas; b) Validation by projection of external samples.

FIG. 22: Example label with the color legend for reading the sensor in case of fish meat.

FIG. 23: Array of sensors for real sample analysis in case of cow's milk.

FIG. 24: Comparison between the sensor colors during degradation at 4° C. of whole milk samples (left), semi-skimmed milk (center) and skimmed milk (right).

FIG. 25: Descriptive models of the degradation process with identification of SAFE, WARNING and HAZARD areas for the three types of milk.

FIG. 26: Example label with the color legend for reading the sensor in case of milk.

FIG. 27: Result of PLS modeling: comparison between true values and calculated values, left, and comparison between true values and calculated values in cross validation.

### DETAILED DESCRIPTION OF THE INVENTION

A first object of the present invention is represented by a system of sensors (array) sensitive to a feature indicative of the freshness of a food product.

In one aspect of the invention, such food product is fresh.

In a favorite aspect, such food product is represented by meat or a fresh meat food.

In a particularly preferred aspect, meat is fresh meat.

For the purposes of the present invention, meat is chicken, beef, pork or fish meat (for example, cod), where other types of meat are not excluded from the present purposes.

Furthermore, meat could be in whole pieces (as chicken breast) or in pieces or slices or minced.

For the purposes of the present invention, meat could be preserved within a package.

Preferably, the meat must be present in an amount of at least 30 grams, preferably 100 grams and even up to 300 grams or more.

For example, for chicken meat is preferably needed an amount of at least around 100-150 grams, for example, for pork is preferably needed an amount of at least around 50-100 grams; for example, for beef is needed an amount of at least around 30-50 grams.

In an even more preferred aspect of the invention, meat may be present within a package in a certain quantity, so that there is a certain ratio between minimum amount of meat and head volume of the package (overlying volume), that is, the free volume of the package not occupied by the meat, in which compounds derived from degradation are released.

In this regard, such ratio could be between around 0.01-0.2 g/cm³, preferably between around 0.02-0.1 g/cm³.

In particular aspects, for chicken meat, such ratio is at least around 0.1 g/cm³, for pork is at least around 0.04 g/cm³ and for beef is at least around 0.02 g/cm³.

According to one aspect of the present invention, the ratio between protein mass and volume of the tray must be around 150 g/500 cm³.

According to a further aspect of the present invention, the food product could be represented by milk.

Milk is intended as fresh food, when subjected to pasteurization procedures or other treatments, of the thermal type or not, in order to prolong the conservation.

According to an alternative aspect of the present patent application, the food product may be represented as a milk-based product, even when subjected to cooking processes.

In particular, the milk may be whole, semi-skimmed or skimmed.

Preferably, such milk is cow milk.

With the term “feature indicative of freshness” it is intended, in particular, the release by the fresh food, or meat, of volatile compounds, in an amount that increases with the progress of its putrefaction process (or degradation).

In general, the degradation process comprises a first phase, in which the attacked substrates are glucose, lactic acid and derivatives thereof, producing, among others, acetic acid and propionic acid, as well as ethanol, while in a second phase the degradation of the protein component occurs, with resulting production of biogenic amines, nitrogen compounds and, at last, sulfur-containing compounds, as thiols.

Those volatile compounds produce pH changes, compared to a fresher food: first towards acidic pH, then returning to a more neutral pH, with simultaneous production of thiols.

Thus, the system of sensors according to the present invention comprises a compound which is sensitive to pH change (to that further reference will be made hereinafter as “indicators”) and/or to the presence (concentration) of thiols or sulfur-containing compounds (to that further reference will be made hereinafter as “binders”).

For the purposes of present patent application, pH variation and concentration of thiols or sulfur-containing compounds is to be understood as referring to the head space of a package containing the meat; indeed, the sensors of the system are not in contact with meat.

For the purposes of the present invention, indicators/binders having certain proprieties may be used, for example:
toning in presence of the analytes of interest,
quick toning in presence of the analytes of interest,
net color change, observable with the naked eye as well,
reproducibility of behavior,
reliability, in such a way to prevent the possibility of false positives or negatives due to the reaction with analytes normally present in the environment, and
presence of a site through which anchoring them to a solid polymer, directly, or through an appropriate derivatization.

In a preferred aspect of the present invention, the indicators selected from the following group may be used: 1-naphthoyl phthalein, thymolphthalein, phenolphthalein, phenol red, m-cresol purple, o-cresol red, bromothymol blue, thymol blue, T-Azo C (disodium salt of 2-[(1H-5-tetrazolyl)azo]-1,8-dihydroxynaphthalene-3,6-disulfonic acid trihydrate), Titan yellow, alizarin yellow, chlorophenol red, bromophenol blue.

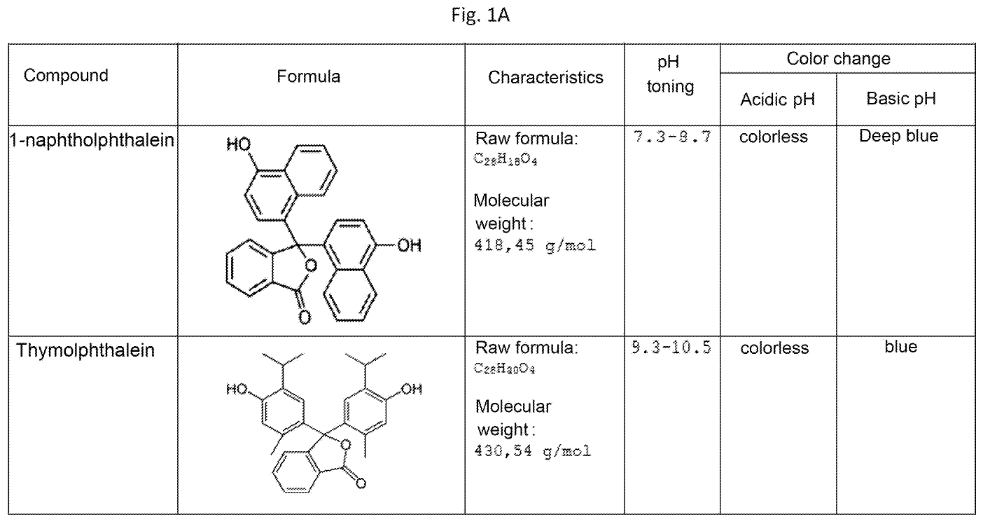

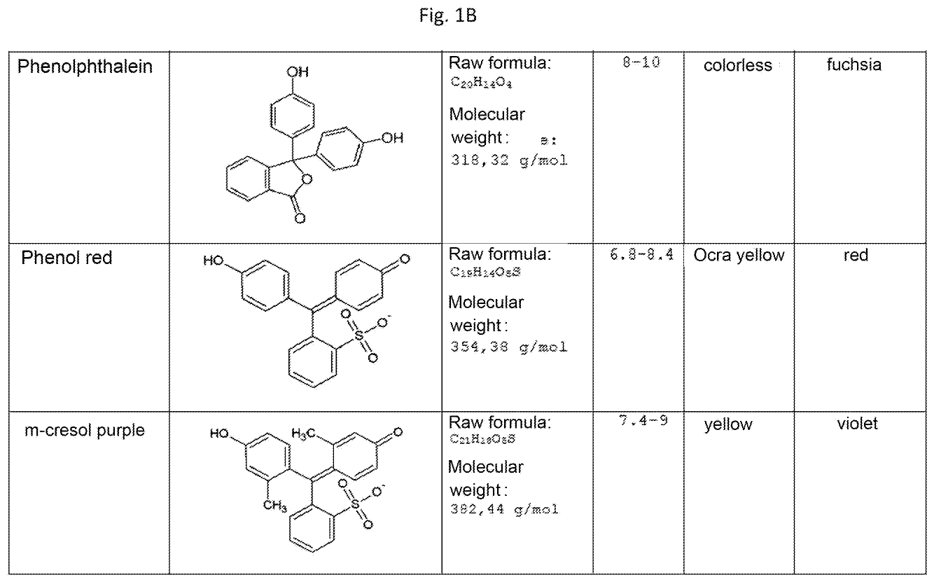

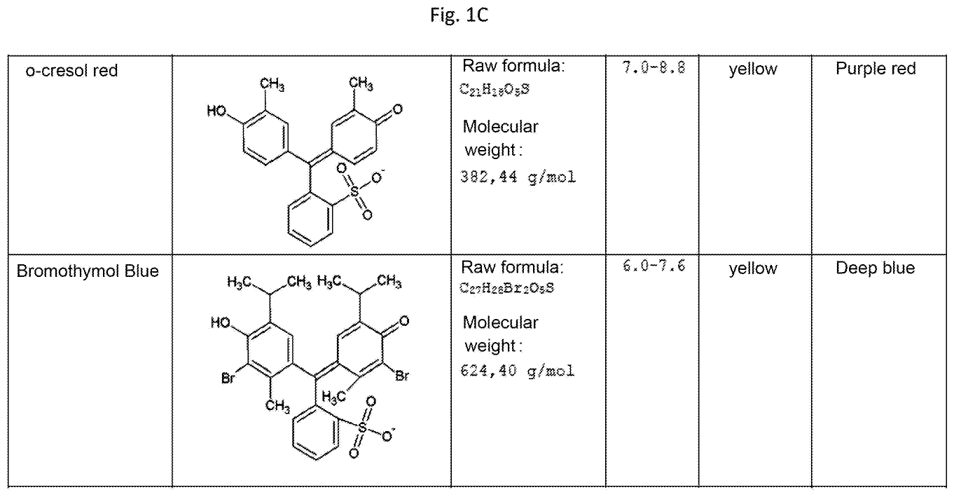

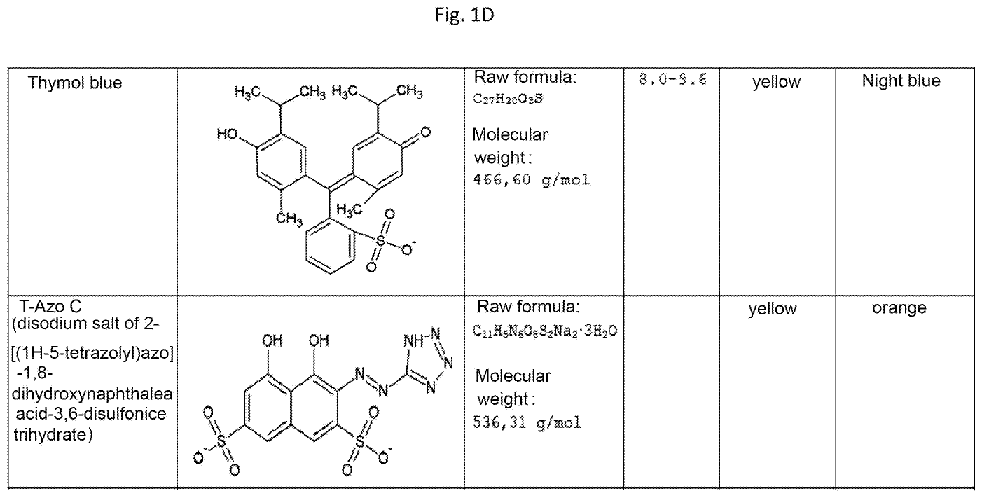

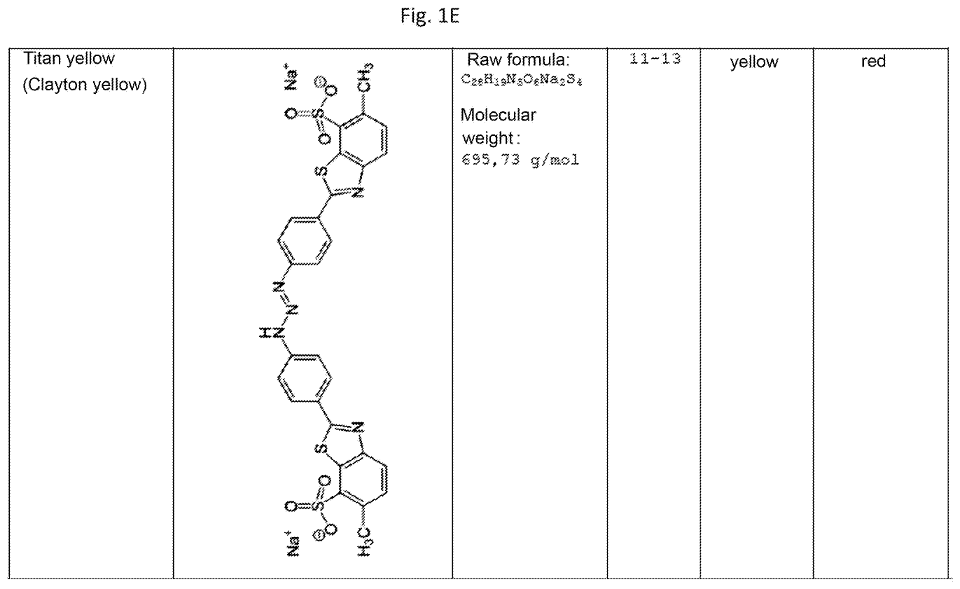

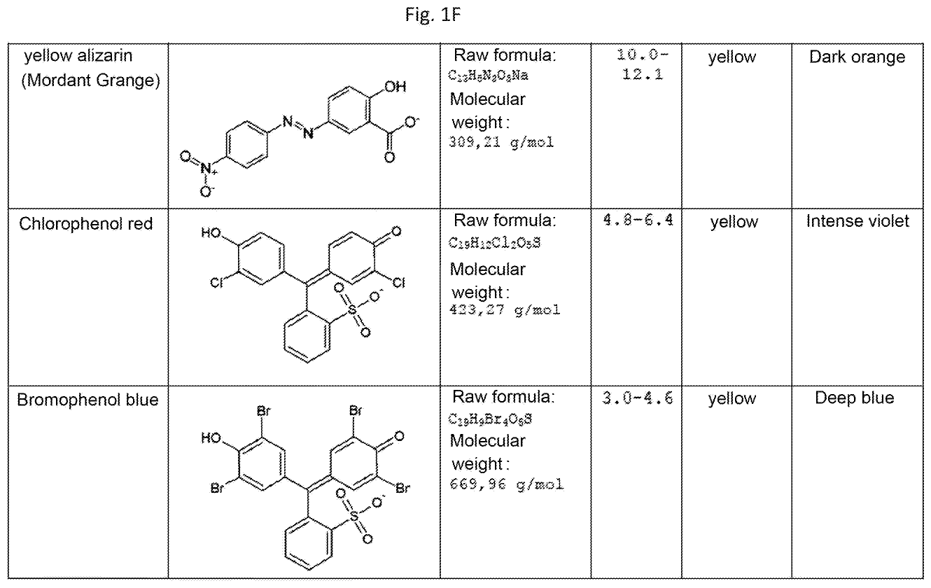

Such indicators have the features reported in FIG. 1.

For the purposes of the present invention, other molecules than those above listed may also be used when they meet the mentioned requirements.

For example, for the indicators that have a phenyl group substituted with a sulfonic group, the use of a structural analog may be contemplated having the sulfonyl group in meta or in para position, instead of ortho position; such modification should lead to steric type advantages, such to allow a better functionalization on a support.

Preferably, such modification could concern m-cresol purple, phenol red, o-cresol red, bromothymol blue, thymol blue, chlorophenol red and bromophenol blue.

In one aspect of the present invention, the indicators that tone to basic or neutral pH are used for the indication of the passage to acidic pH caused by the release of acidic compounds that characterize the former phase of the degradation of meat; for this purpose, these indicators are used in their basic form.

In particular, the indicators are: m-cresol purple, o-cresol red, bromothymol blue, thymol blue, 1-naphtholphthalein, thymolphthalein, phenolphthalein, phenol red, T-azo-C, Titan yellow and Alizarin yellow.

More preferably, the indicators are: m-cresol purple, o-cresol red, bromothymol blue, thymol blue, chlorophenol red and bromophenol blue.

In another aspect of the present invention, the indicators that tone to weakly acidic pH are used for the indication of the passage to basic pH, when, thus, the production of acids ends; for this purpose, these indicators are used in their acidic form.

In particular, the indicators are: chlorophenol red, bromophenol blue and, more preferably, chlorophenol red.

Binders

For the purposes of the present invention, binders for the analytes of interest may be used.

Specifically, those analytes are represented by thiols.

For this purpose, Ellman's reagent (2,2′-dinitro-5,5′-dithiodibenzoic acid (DTNB)) may be used, for which it is particularly selective:

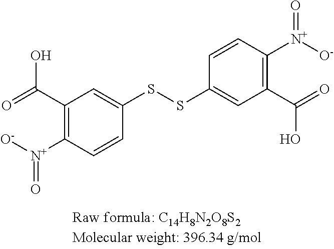 [SMILES: O=C(O)c1cc(SSc2ccc([N+](=O)[O-])c(C(=O)O)c2)ccc1[N+](=O)[O-]]

The breakdown of the molecule leads to the release of chromophore 5-thio-2-nitrobenzoate (TNB), characterized by an intense absorption at 412 nm, and by yellow color.

As above described, the present invention relates to a system of sensors, meaning a set of two or three sensors.

This set has the advantage of providing a more precise indication of the freshness of the meat product.

According to a first aspect of the invention, such set comprises a sensor sensitive to pH change and a sensor sensitive to the presence of thiols.

In particular, such sensor may be a sensor comprising an indicator toning the color to acidic pH.

According to one aspect invention, such sensor comprises an indicator that may be used in the acidic or basic form.

In a preferred aspect of the invention, the system comprises three sensors, two of which include indicators sensitive to pH change and one comprises a binder sensitive to the presence of thiols.

In another preferred aspect of the invention, the system comprises two sensors, both sensitive to pH change (thus, the sensor sensitive to the presence of thiols or sulfur-containing compounds is not included).

More particularly, of two sensors sensitive to pH change, preferably one is used in its basic form and highlights the production of acidic substances, as it changes color from basic to acidic color in their presence.

This sensor is therefore indicative of the first phase of degradation.

The second sensor, instead, is preferably a sensor put in its acidic form, which has such a protonation constant to detect the first partial pH increase that takes place when no more acidic substances are released, and it will change its color from acidic color to the basic color.

In a preferred aspect of the invention, the first sensor may be represented by chlorophenol red or bromothymol blue.

In another preferred aspect of the invention, the two sensors sensitive to pH change are represented by chlorophenol red and bromothymol blue; such aspect results particularly preferred for the application to chicken meat.

In a further preferred aspect of the invention, these two sensors can be used in combination with a sensor sensitive to thiols, comprising, as binder, Ellman's reagent.

That combination turned out to be especially useful in indicating the degradation status of the chicken meat.

To complete, the system of sensors can be provided with reference means (or control elements) represented by elements with one or more portions having a color respectively corresponding to one or more of the colors that the sensor acquires with the varying of pH or of the concentration of thiols or sulfur-containing compounds.

In particular, such color of the control elements may correspond to a pH value or to a concentration of thiols or sulfur-containing compounds previously correlated with one or more of the degradation statuses of the meat: safe, warning or hazardous to health.

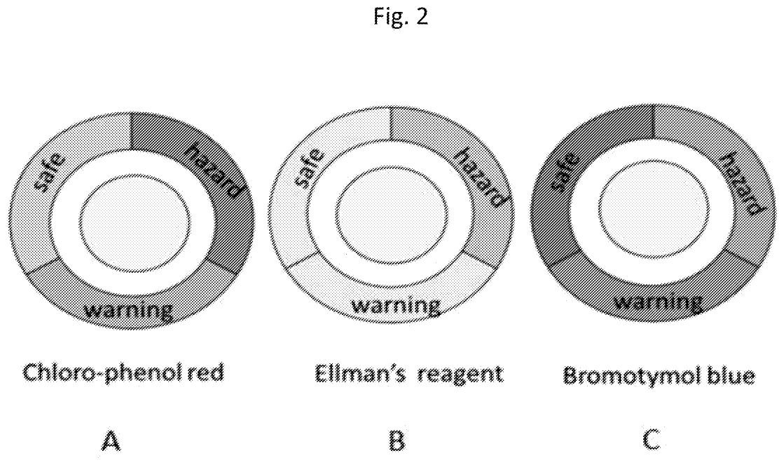

For example, as represented in FIG. 2, depending on the pH change and/or on the development of sulfur compounds, the sensor will acquire a color that, thanks to the comparison with the control elements, may be recognized as representative of a security “SAFE”, attention “WARNING” or danger “HAZARD” situation.

With the purpose of being used for preparing a sensor, the indicators and/or the binders above described are immobilized or adsorbed on a support material.

As regards immobilization, this happens through covalent binding.

Thus, an indicator or a binder immobilized on a support according to the present invention represents per se an object of the present patent application.

According to a preferred aspect, the support material is represented by a material suitable for food packaging and, in particular, for meat and meat food products.

In particular, in a first aspect, said support may be represented by a polymeric material support.

In particular, such polymer may be selected in the group comprising: ethylene-vinyl-alcohol (EvOH), in particular, EvOH 27%, EvOH 29%, EvOH 32%, EvOH 38% or EvOH 44%, ethylene vinyl acetate (EVA), linear low density polyethylene modified with maleic anhydride (Orevac® 18362).

According to a further aspect of the invention, the support may be represented by carboxymethyl cellulose (CMC).

According to alternative aspects, the support may be represented by a fabric or a non-woven fabric.

Such fabric and non-woven fabric may be of natural or synthetic fiber.

The process with which the polymeric material support as above described is functionalized with the indicators/binders described, obtaining in this manner an immobilized sensor, represents a second object of the present invention.

In particular, this process comprises the steps of:

1) functionalizing an indicator or a binder according to the present invention,

2) derivatizing a polymeric material according to the present invention with the indicator or the functionalized binder obtained from step 1).

In particular, regarding step 1), this comprises preparing a solution of indicator/binder according to the above described in an appropriate solvent and its reaction with an agent able to introduce a functional group in the indicator/binder.

In a preferred aspect, the group introduced is a halide group and, preferably, a chloride.

In this regard, then, a chlorinating agent may be used, selected from the group comprising: thionyl chloride, phosphorus pentachloride, phosphorus oxychloride, bis(trichloromethyl)carbonate, phosphonitrilic chloride trimer, trichlor triazine, oxalyl chloride, chlorosulfonic acid, N-chlorosuccinimide.

As regards to solvents, one of these is for example represented by dimethylformamide.

In the case in which the support for immobilization is represented by a polymer, in step 2) a solution of the polymer is previously prepared to be derivatized in an appropriate solvent.

In this regard, N,N-dimethylacetamide (DMA) may be used.

Step 2) is then conducted adding to the solution of the polymer to be derivatized the indicator or functionalized binder solution.

For the purposes of the present invention, after step 2) the derivatized polymer is precipitated in an appropriate solvent (step 3).

In this regard, then, a precipitating solvent may be used, selected from the group comprising: carbon tetrachloride, chloroform, benzene, toluene, xylene, ethyl acetate, tetrahydrofuran, water, acetone and dichloromethane.

With the precipitation, then a polymer derivatized in solid form is obtained, which is collected by filtration and further dried, so as to obtain a solid glassy form material.

For the purposes of the present invention, the support of polymeric nature is in the form of a film that may be obtained by techniques known in the sector, as, for example, extrusion.

Alternatively, the polymeric film may also be obtained with a heated hydraulic press.

For the derivatization of the carboxymethyl cellulose support we may proceed in analogy with the process for preparing the polymeric support.

If the support is not of polymeric nature, as described above, an adsorption process is enough.

In particular, for this aspect, said indicator or binder is used in an amount of around 1-2% of the maximum capacity of said indicator.

According to a preferred embodiment of the invention, the support of the system of sensors is represented by a polymeric material, preferably in the form of a film.

This polymeric film is more preferably represented by a polymeric film in EvOH, as described above.

According to an even more preferred embodiment, the system of sensors comprises the indicators represented by: bromothymol blue, thymol blue and bromophenol blue.

According to an even more preferred embodiment, the system of sensors comprises an EvOH polymeric film and indicators represented by: bromothymol blue, thymol blue and bromophenol blue.

According to a further preferred embodiment, the system of sensors comprises an EvOH polymeric film and indicators represented by: bromothymol blue, thymol blue and bromophenol blue and is suitable for a fresh food represented by beef, chicken or fish.

According to an alternative embodiment, the support of the system of sensors is represented by a polymeric material, preferably in the form of a film.

This polymeric film is more preferably represented by a polymeric film in EvOH, as described above.

According to an even more preferred embodiment, the system of sensors comprises bromothymol blue indicator.

According to an even more preferred embodiment, the system of sensors comprises an EvOH polymeric film and bromothymol blue indicator.

According to a further preferred embodiment, the system of sensors comprises an EvOH polymeric film and an indicator represented by bromothymol blue and it is suitable for a fresh food represented by milk.

For the purposes of the present invention, the system of sensors above described may to be comprised in a package or in container or in an element of a package or container, for example a lid or a film for closing a tray, for meat or for meat food, or for a milk container.

Such package or such element of a package or container represent further objects of the present invention.

In particular, the position of sensors and the proximity to fresh food will be such to allow the system to work and the consumer to observe the sensors status.

It is clear that the control elements may be intended as part of the system of sensors, as above described, or as part of the container or part of the container.

According to a further object of the invention, a system of sensors as above described may be used as litmus paper.

For such application, the support of the system of sensors is represented by a polymeric material, possibly in the form of film.

The invention will be here described below with particular reference to some examples to be considered as non-limiting.

### Example 1

### Preliminary Phase on Ion Exchange Support

Tests with Synthetic Samples

The molecules, adsorbed on different solid phases, have been exposed to the vapors of specific solutions, within tightly closed and transparent boxes, so as to evaluate responsiveness and sensibility. Ammonia and acetic acidic solutions have been used for pH indicators and ethanol for Ellman's reagent. The sensitivity of the molecules was also evaluated by varying the concentration of the underlying solutions from 1 M to 0.01 M for acetic acid and 0.0001 M for ammonia.

Following such tests, the most promising molecules have been selected for setting-up the final array: for pH indicators, the ones able to recognize the presence of either acidic and basic species up to the lowest concentrations have been selected while Ellman's reagent has been confirmed, once high sensitivity was verified.

Setting-Up the Array

The definitive array consists of six spots onto which the following molecules are adsorbed:

M-cresol purple (1),

O-cresol red (2),

Bromothymol blue (3),

Thymol blue (4),

Chlorophenol red (5) and

Ellman's reagent (6).

Array optimization is obtained by evaluating the following characteristics, before proceeding with the analysis of real samples:

Amount of Binder Adsorbed:

To determine the optimal amount, the maximum capacity of the solid phase is calculated, and the sensitivity of the spots with different adsorbed amounts is evaluated. It is verified that the larger sensitivity is shown by the spots with less adsorbed amount since the amount of acid, or base, needed for the complete toning is proportional to the amount of adsorbed indicator. At the same time, however, if the amount is too small, the color is very faded and, with the naked eye, the toning is not well understood.

Therefore, the optimal amount is around 1-2% of the maximum capacity for all indicators.

Equilibration Time:

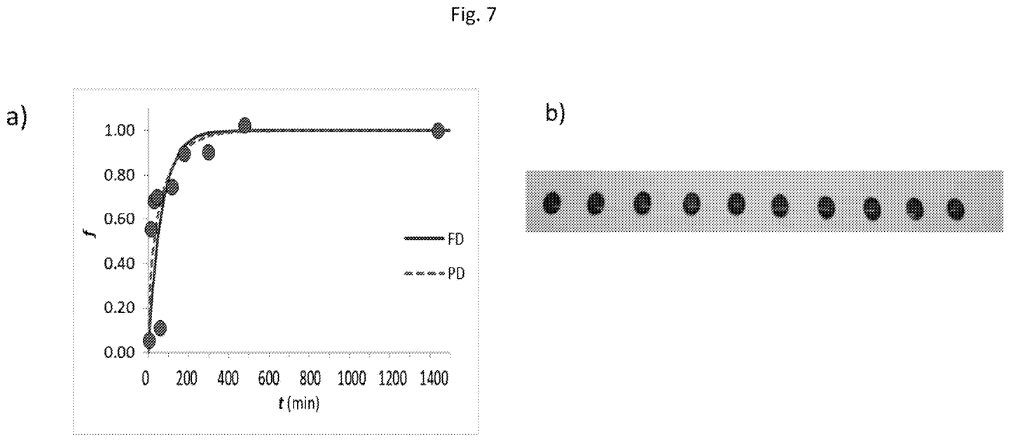

Adsorption kinetics of molecules on the solid phase are studied and it is verified that the process adsorption, in the operative conditions used, is completed after 6-7 hours (FIG. 7a).

Color Reproducibility:

10 independent spots for each molecule are prepared (FIG. 7B), the adsorbed indicator is quantified, and the obtained value is compared with the true one. It is verified that the two values are equal to a confidence level of 95%.

Initial pH:

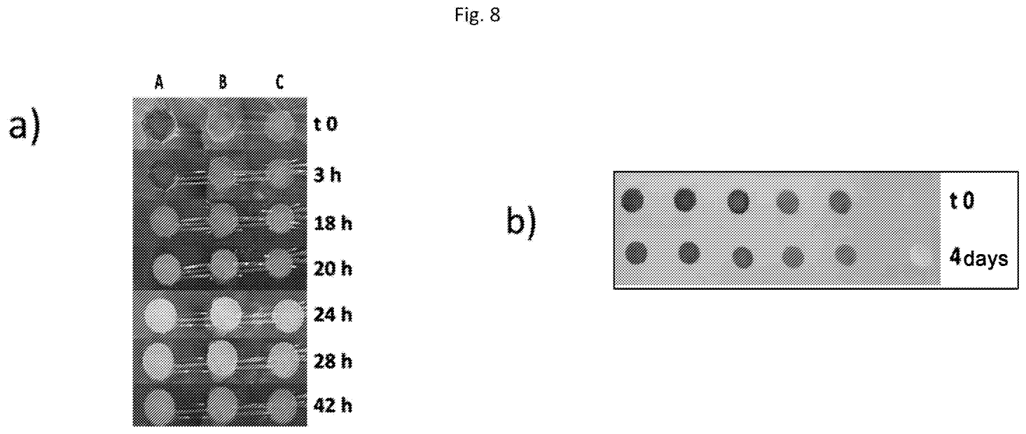

To maximize the sensitivity of the array against the degradation products output from the meat it is necessary to evaluate, for each indicator, the best initial pH, and therefore the best color. For this reason, sensors are prepared at different pH and are set out to the same samples of chicken meat to verify, for each molecule, which one guaranteed the best performances (FIG. 8a).

Suspension Mode:

Several suspension modes are also evaluated of the spots within the trays used for meat retail sale. It is necessary that the meat does not comes in contact with the array and that the support used is inert and does not cause the toning of the indicators. As definitive mode, the use of a matte scotch tape is chosen.

Blank Analysis:

As last step, it is necessary to verify that the sensors do not show a toning in absence of degradation products of the meat, so as to prevent incorrect attributions. For a commercial application, the false positives, that is, fresh samples evaluated as degraded by the array, and above all the false negatives, i.e. no-longer-edible not-recognized samples, are indeed harmful. For these analyses, several arrays are exposed to air and to a phosphate buffer solution, commonly used to simulate biological matrices, and it is noted that the array remains unchanged up to 4 days (FIG. 8b).

Setting the Tests with the Real Sample

Meat samples are purchased at the local supermarket, the same day of delivery, in such a way to work with initial samples at the same degree of freshness.

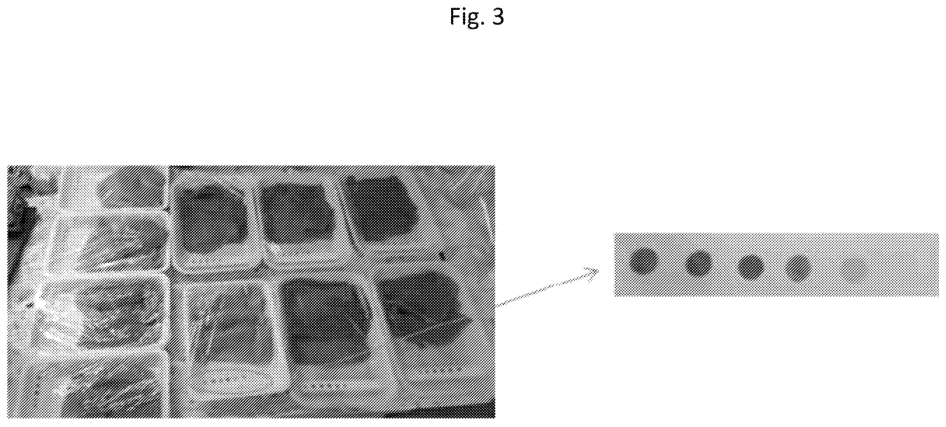

Based on these premises, the indicators that tone to a basic or neutral pH (1-4), prepared in basic form, are used to recognize the acidic compounds characterizing the first degradation phase, through transition to the acidic color; the indicator that tones to a weakly acidic pH (5) is prepared in acidic form and transitions to basic color when the production of acidic compounds ends. As for instead Ellman's reagent, it colors intensely in the second degradation phase, in presence of volatile thiols. An example of the final array is shown in FIG. 3.

Finally, with regard to data acquisition, photographs of the array are taken during the full degradation process, in conditions of constant light, with the camera of a smartphone, through the transparent film, so as to not open the package and do not allow an exchange with the outside air. With this procedure, the samples of chicken meat, beef, pork and cod fillets are analyzed.

Monitoring of Chicken Meat

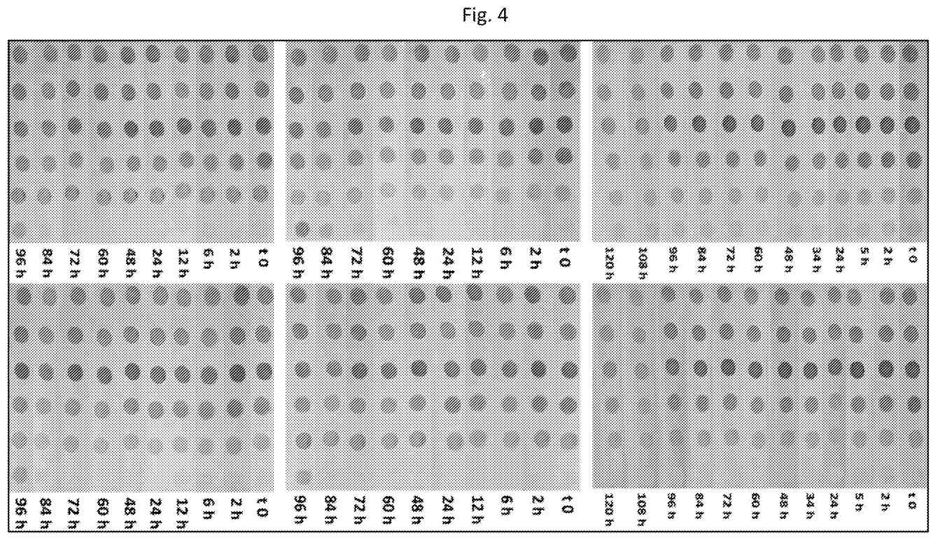

FIG. 4 shows the comparison of the sensor colors during the degradation monitoring in fridge.

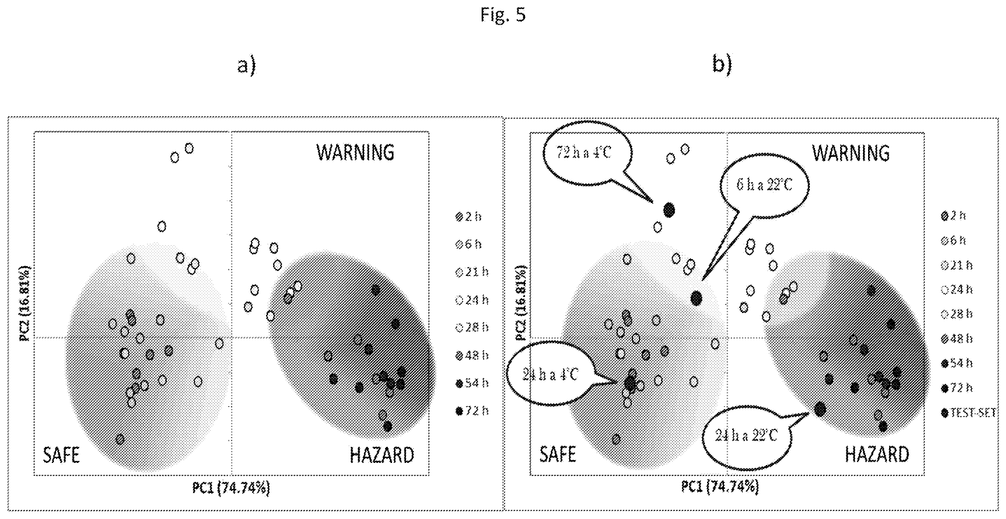

RGB codes of the individual spots of the array are sampled every time and the processing of those data is carried out with the chemometric PCA, Principal Component Analysis, technique which allows to divide the degradation process in three main phases, called SAFE, WARNING and HAZARD, as shown in FIG. 5a.

The model obtained is validated by projecting within the unknown samples so as to verify the correct attribution. See, for this purpose, FIG. 5b, in which the samples preserved in different way are assigned in a correct way compared to what is expected from the observed evolutions inside and outside the fridge. Mostly, the model is validated through independent instrumental analyses, who want to prove that the clusters identified by the PCA match different degrees of degradation. For such purpose we proceed as described below.

Validation of Color Attributions to Degradation Phase

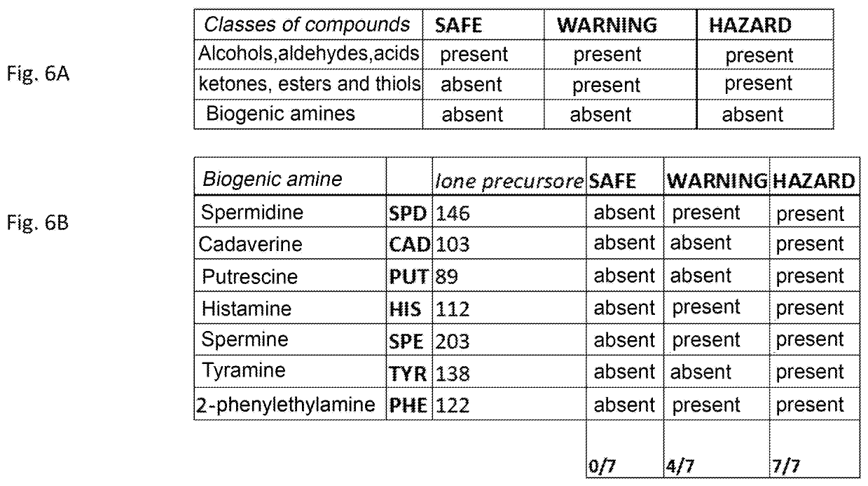

Analyses are performed in the CGS laboratory, Centro Grandi Strumenti, at UniPv, which carries out third-party analyses. Samples taken in the head space, and samples taken in the meat underneath, have been subjected to analysis. These samples are subjected to two standard analysis procedures, one directly on the gas drawn in head space, the other after appropriate attack of the matrix and extraction of substances of interest, according to a procedure as per literature (Sirocchi, Caprioli, Ricciutelli, Vittori, & Sagratini, 2014)]. In FIG. 6a, the compound classes identified by HSSPME-GC/MS in head space of samples identified as SAFE, WARNING and HAZARD, are reported. Note how biogenic amines are never found, as previously supposed.

As for the identification of amines in meat, on samples prepared and preserved precisely like those used for analysis in the test space, we proceed with a TCA extraction from the properly mashed material, with elution of this extract and HPLC-ESI/MS analysis. The results are reported in FIG. 6B.

As noted from FIG. 6b, the biogenic amines are present inside the meat, all seven in the meat classified as HAZARD, some in that classified as WARNING, and absent in the samples of meat classified as SAFE. The analyses confirm that the substances present in the head space and in the meat are different in the three degradation phases. These results also demonstrate, beyond all doubt, that the biogenic amines are actually produced at some point in degradation, within the meat, but these never go into head space, not even in advanced degradation phases, due to buffered pH typical of biological matrices. These analyses confirm that biogenic amines are not found and that the classification based on the colors of the volatile degradation products is not an artifact, but allows to correctly identify the decomposition stages, although it is not at all related to the production of biogenic amines.

Monitoring of Beef

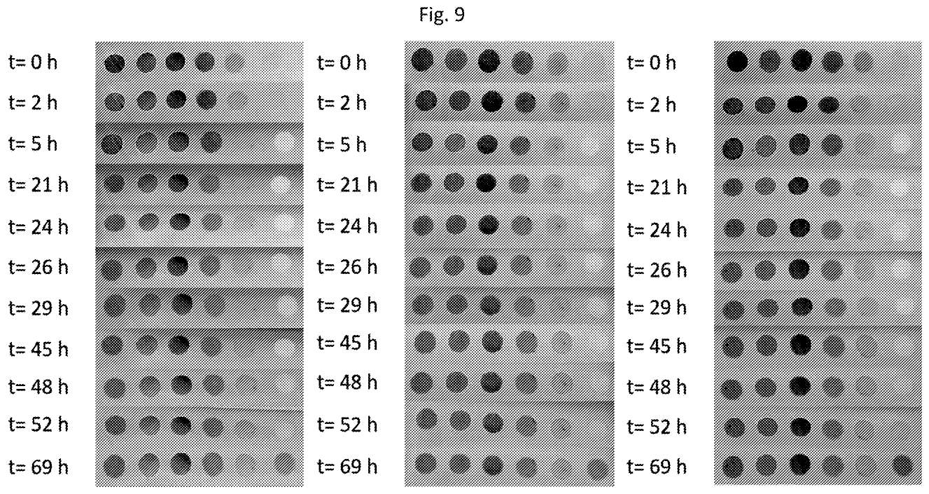

In FIG. 9 the comparison of the sensor colors during the degradation monitoring at room temperature is reported.

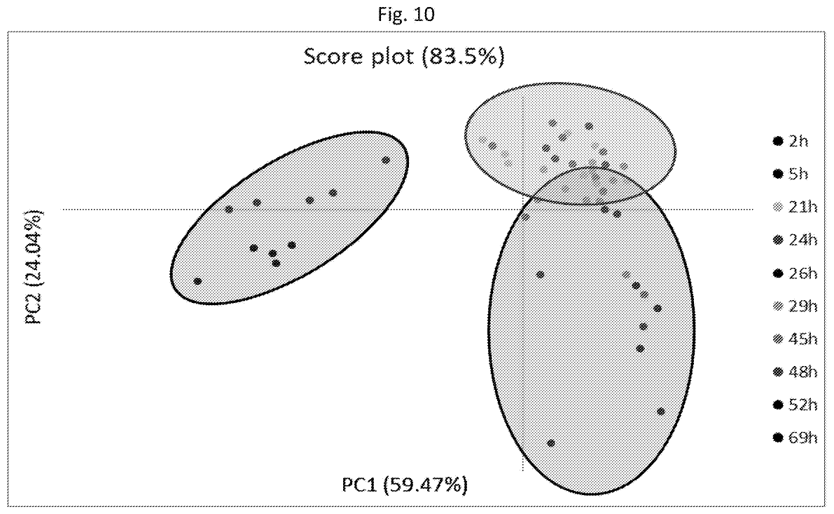

RGB codes of the individual spots of the array are sampled at every time and the processing of those data is carried out with chemometric PCA, Principal Component Analysis, technique which allows divide the degradation process in three main phases, called SAFE, WARNING and HAZARD, as shown in FIG. 10.

The model confirms the lower perishability of this fresh food compared to chicken meat, and even the different duration of the degradation phases. Despite this, the proposed array allows to adequately monitor the degradation of such fresh food, being enough versatile.

Monitoring of Pork

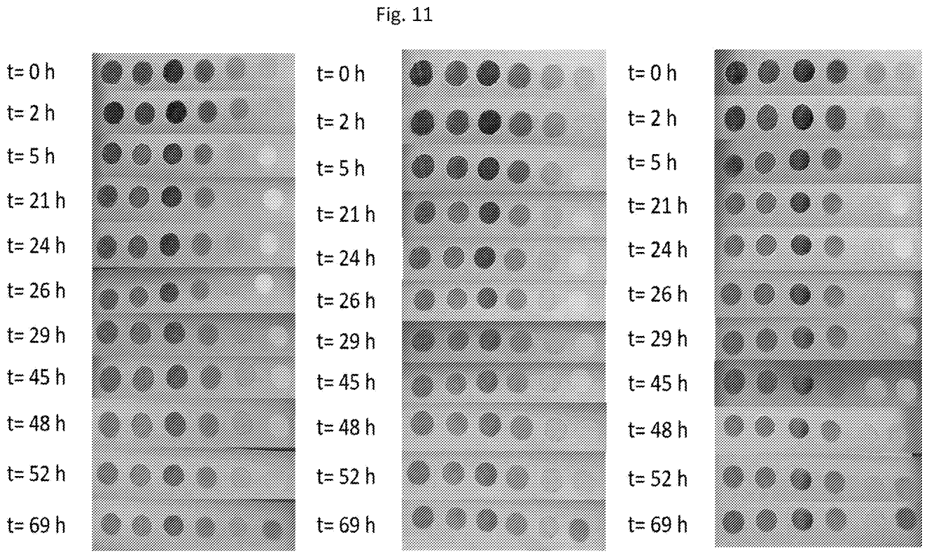

In FIG. 11 the comparison of the sensor colors during the degradation monitoring at room temperature is reported.

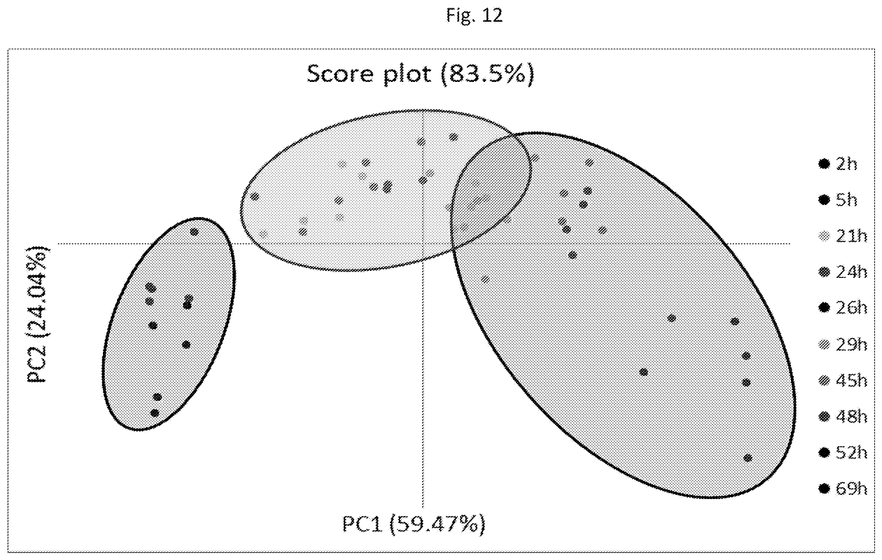

RGB codes of the individual spots of the array are sampled at every time and the processing of those data is carried out with chemometric PCA, Principal Component Analysis, technique which allows divide the degradation process in three main phases, called SAFE, WARNING and HAZARD, as shown in FIG. 12.

The degradation of the pork is similar to that of beef, in terms of perishability and duration of the degradation phases.

Monitoring of Cod Fillets

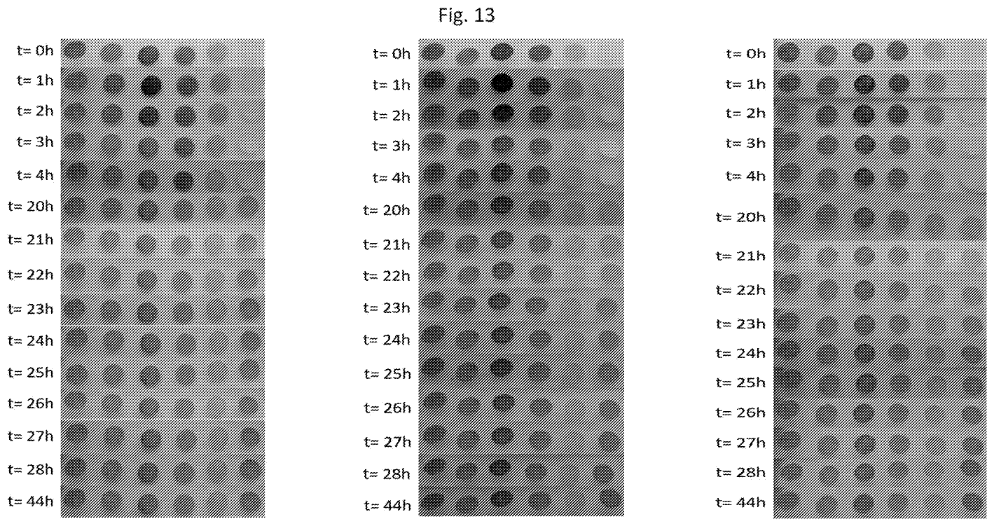

In FIG. 13 the comparison of the sensor colors during the degradation monitoring at room temperature is reported.

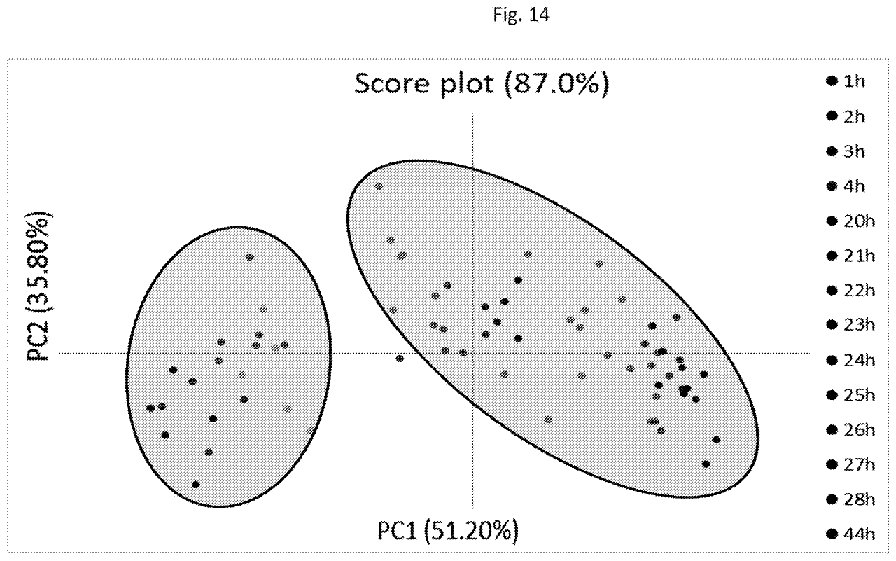

RGB codes of the individual spots of the array are sampled at every time and the processing of those data is carried out with chemometric PCA, Principal Component Analysis, technique which allows divide the degradation process in three main phases, called SAFE and HAZARD, as shown in FIG. 14.

As expected, the degradation of cod fillets is much faster than observed with the meat. Moreover, at room temperature, only two steps are distinguishable and separable in extremely clean way.

As last preliminary test, always thinking to a commercial application, in which the meat packages do not all contain the same mass of samples, sensitivity tests have also been carried out to check the minimum sample mass to which the array shows the same trend, during degradation. For all meats, the minimum allowed value is around a quarter of the mass normally contained. Therefore the device, as proposed here, has an evolution of color that attributes the phases in adequate way up to a protein matter mass/volume ratio of the tray equal to 150 g/500 cm³.

### Example 2

### EvOH Derivatization with Organic Dyes

1-Chlorination of Bromothymol Blue

Reagents
bromothymol Blue 100 mg
Thionyl chloride SOCl₂ 10 mL

### Method

In a 25 mL (perfectly anhydrous) flask a binder solution (100 mg) in 10 mL of thionyl chloride is prepared. The mixture thus obtained is reflux-heated for 3 hours, maintaining a moderate boiling. After this time, the solution is cooled at room temperature for a few hours and the excess of thionyl chloride removed by evaporation at reduced pressure, obtaining a residue that is used as it is.

### 2-EvOH Functionalization

Reagents
Chlorinated bromothymol Blue
EvOH
NaOH
DMA
CH₂Cl₂

Method

In a three-necked flask EvOH is dissolved in DMA, warming at 100° C., shaking with magnetic stirrer and keeping the solution under N₂ flow. Once dissolved the polymer, NaOH, previously dissolved in DMA, is added. We then proceed to add chloride of the organic dye.

The dye is dissolved in DMA, just before adding it to the solution containing the polymer. The solution of the chlorinated binder thus obtained is slowly added dropwise via drip funnel.

At the end of additions, we continue heating for around 4 h, then the mixture is allowed to cool in N2 flow, earlier at room temperature and later in ice.

The cold mixture is poured in methylene chloride, under vigorous stirring, with consequent precipitation of the functionalized polymer, which is collected by filtration on Buchner funnel.

The solid, washed with additional methylene chloride and dried for 18 hours on filter, is removed and further dried with Abderhalden pistol for a day at 60-80° C. in order to get the end product as a glassy solid.

3-Scale-Up Process

In the table below, a scheme of an example of scale-up adopted in the tests performed up to here is reported, in the instance of the polymer-based sensor with Bromothymol blue binder from starting 5 g up to 150 g for the Bromothymol-based polymer. Further scale-up is possible with suitable instrumentation.

| m | V DMA | M | V DMA | m | V DMA | V DMA | |
|---|---|---|---|---|---|---|---|
| EvOH | EvOH | NaOH | NaOH | Blue | Blue | tot | V CH₂Cl₂ |
| (g) | (mL) | (mg) | (mL) | (g) | (mL) | (mL) | (mL) |
| 5 | 150 | 10 | 30 | 0.1 | 20 | 200 | 300 + 200 |
| 10 | 150 | 20 | 50 | 0.2 | 40 | 240 | 500 + 300 |
| 15 | 250 | 30 | 50 | 0.35 | 50 | 350 | 650 + 400 |
| 150 | 700 | 400 | 100 | 3.5 | 100 | 900 | 1500 + 500 |

4-Polymeric Film Preparation

The synthesis-obtained glassy polymer is later chopped and pressed to get uniform polymer films, suitable for analytical measurements.

The pressing is carried out using a heated hydraulic press: the previously chopped polymer (300 mg) is distributed between the two plates of the press and brought to temperature. Once the temperature is stabilized, a suitable force is applied for a previously optimized period to get the final polymeric film. The working temperature depends from the binder with which the polymer was functionalized and is evaluated by DSC analysis of the functionalized polymer. The pressure applied and the duration of the application depend instead on the chemical-physical characteristics of the starting polymer, in particular from ethylene % and from MFR (Melt Flow Rate).

From the films thus obtained, miniature circular sensors with a diameter of 0.5 cm are obtained, used for all subsequent tests.

Following the same mode of the Example, with appropriate adaptations within the reach of the person skilled in the art, additional sensors can be prepared using other chlorinating agents, solvents, functionalization solvents and types of polymer.

Chlorinating Agents:
Thionyl chloride SOCl₂
Phosphorus pentachloride PCl₃
Phosphorus oxychloride POCl₃
Bis(trichloromethyl)carbonate (CCl₃C)₂CO
Chloride phosphonitrile trimer Cl₆N₃P₃
Trichlor triazine C₃Cl₃N₃
Oxalyl chloride C₂O₂Cl₂
Chlorosulfonic acid HSO₃Cl
N-chlorosuccinimide C₄H₄ClNO₂

Solvents for Functionalization:
DMF

Solvents for Precipitation:
Carbon tetrachloride
Chloroform
Benzene
Toluene
Xylene
Ethyl acetate
Tetrahydrofuran
Water

Commercial EVOHs
27%
29%
320
38%
44%

Other Polymers for Food Packaging
Ethylene vinyl acetate EVA
Orevac 18362®

### Example 3

### Derivatization of Carboxymethyl Cellulose (CMC) with Organic Dyes

1-Chlorination of the Tymol Blue

Reagents
Thymol Blue 50 mg
Thionyl chloride SOCl2 10 mL

Method

In a 25 mL (perfectly anhydrous) flask a binder solution (100 mg) in 10 mL of thionyl chloride is prepared. The mixture thus obtained is reflux-heated for 3 hours, maintaining a moderate boiling. After this time, the solution is cooled at room temperature for a few hours and the excess of thionyl chloride removed by evaporation at reduced pressure, obtaining a residue that is used as it is.

2-Functionalization of CMC

Reagents
Chlorinated Thymol Blue
CMC
Toluene
CH₂Cl₂

Method

In a two-necked flask CMC is suspended in toluene, heating at 65° C., shaking with magnetic stirrer. Once reached the temperature, we then proceed to the addition of chloride of the organic dye.

The dye is dissolved in toluene, just before adding it to solution containing the CMC. The solution of the chlorinated binder thus obtained is slowly added dropwise via drip funnel. At the ends of additions, the heating goes on for around 3 h, then the suspension is allowed to cool, before at room temperature and later in ice.

The cold suspension is filtered on Buchner filter, the solid obtained is washed with 30 mL of methylene chloride and dried for 18 hours on filter.

3-Preparation of the Sensor

Reagents
Functionalized CMC
Glycerol
Starch
H₂O

Method

An aqueous solution containing 5% (w/V) of functionalized CMC and glycerol, and 2% (w/V) of starch is prepared, by hot-dissolving the glycerol and starch under stirring, and then adding the functionalized CMC in small quantities. Once a transparent mixture is obtained, it is poured in a Petri dish of suitable diameter and placed in oven at 50° C. for around 4-5 h. Then, the film is allowed to cool at room temperature and removed from the dish.

Alternatively, it is possible to prepare a sensor already supported on solid inert material, depositing 50 μL of the previously described mixture on a cellulosic base support. After the same heating treatment, miniaturized sensors already supported on inert material are obtained.

Following the same mode of the Example, with appropriate adaptations within the reach of the person skilled in the art, additional sensors can be prepared using other chlorinating agents, solvents, functionalization solvents and types of polymer.

Chlorinating Agents
Thionyl chloride SOCl₂
Phosphorus pentachloride PCl₃
Phosphorus oxychloride POCl₃
Bis(trichloromethyl)carbonate (CCl₃C)₂CO
Chloride phosphonitrile trimer C₁₆N₃P₃
Trichlor triazine C₃Cl₃N₃
Oxalyl chloride C₂O₂Cl₂
Chlorosulfonic acid HSO₃Cl
N-chlorosuccinimide C₄H₄ClNO₂

Solvents for Functionalization
DMF
Methylene chloride

Solvents for the Wash
MeOH
EtOH
Toluene

### Example 4

### Polymeric Support

The sequence of analysis, setting-up and optimizations described in Example 1 is repeated on polymer base sensors, obtained with the method described in Example 2.

Several tests with synthetic samples, appropriately prepared in laboratory, and monitoring of different foods with consequent validations have been carried out.

Tests with Synthetic Samples

The sensors, consisting of the polymeric support appropriately derivatized with the indicators listed above, have been tested for evaluating the toning kinetics in different conditions and the sensitivity to volatile analytes at decreasing concentrations. For kinetic tests, the sensors have been balanced at acidic or basic pH and then immersed in appropriate solutions. the sensors were examined at predetermined times by means of colorimetric analysis and data processing was carried out with appropriate chemometric techniques. Such analysis was carried out with solutions at 0.10 M and 0.01 M concentrations of NaOH and HNO₃ and with pH 7 (0,008 M) phosphate buffer. For instead evaluating the sensitivity to volatile analytes, the sensors have been balanced to appropriate pH and then set out to specific solution vapors, within tightly closed and transparent boxes. Ammonia solutions and acetic acid have been used at 1.00 M, 0.10 M, 0.01 M and 0.001 M concentrations. The same analysis was carried out also using pH 7 (0.008 M) phosphate buffer to verify that the indicators did not show an evident color change following the exposure to air. In this manner, it shows that the color change, observed during the exposition to the same over the fresh foods, is not due to reaction with air but instead with the degradation volatile by-products.

Following such tests, the most promising molecules have been selected for setting-up the final array: the pH indicators chosen were those able to recognize the presence of weakly acidic or basic species up to the lowest concentrations, in parallel with what already verified for the other support. In this version of the array, Ellman's reagent was instead excluded because, although derivatization has succeeded, the polymeric support turned out not sufficiently permeable to allow thiol detecting.

Setting-Up of the Array

The definitive array consists of six sensors, derivatized with special pH indicators:
Purple m-cresol (1),
Red o-cresol (2),
Bromothymol blue (3),
Thymol blue (4),
Chlorophenol red (5) and
Bromophenol blue (6).

The choice of the best receptors is closely linked to the solid phase in use. Anchoring of a molecule to a solid phase in fact causes slight changes in its behavior as acidic-basic indicator or in the color developed or, as in this case, in the toning pH range. For this reason, with the pH range useful for the recognition of degradation by-products being known, it is appropriate to select, for each solid phase, the indicators that change color in that range.

To standardize the preparation of those sensors, the following characteristics were evaluated:

Initial pH:

Since the polymer sensors are extremely sensitive, a full or partial toning is observed starting also from highly acidic or basic pHs. Working in this manner, moreover, the color change is very clear and easily recognizable with the naked eye. Therefore, it is chosen to balance the sensors in NaOH 0.10 M or 6 in HNO₃ 0.01 M, according to fresh food of interest (see below).

Equilibration Time:

From previous kinetic studies we derive that the sensors, in conditions reported in the previous point, are balanced after 2 h.

Color Reproducibility:

Considering 10 sensors for each receptor, we analyze the color and the variability between the sensors both in original form and after equilibration to the pH of interest, for the tests on real sample.

Setting the Tests with the Real Sample

The operating modes for tests with the real sample are the same used in the instance of the preliminary tests above described in relation to the synthetic sample, as unchanged is also the procedure of capturing and processing the photographs of the sensor. Compared to these tests, however, the choice of the fresh food onto which to test the sensor operation has been widened: in particular, samples of sliced chicken breast, cod fillets and cow's milk of different types (whole, semi-skimmed and skimmed) were used. For each food matrix is necessary to adapt the preparation of sensors, in particular, the initial pH, and the suspension mode.

Monitoring on Chicken Meat

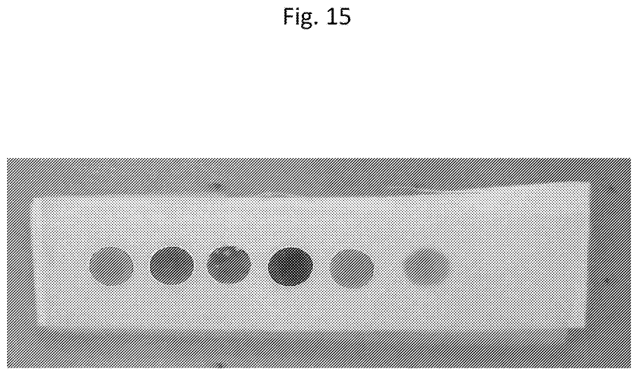

In this case, the indicators toning to a basic or neutral pH (from 1 to 5), prepared in basic form, are used for recognition of acidic compounds, characterizing the first degradation phase, through passage to acidic color; the indicator that tones to a weakly acidic pH (6) is prepared in acidic form and transitions to basic color when the production of acidic compounds ends. An example of the final array is shown in FIG. 15.

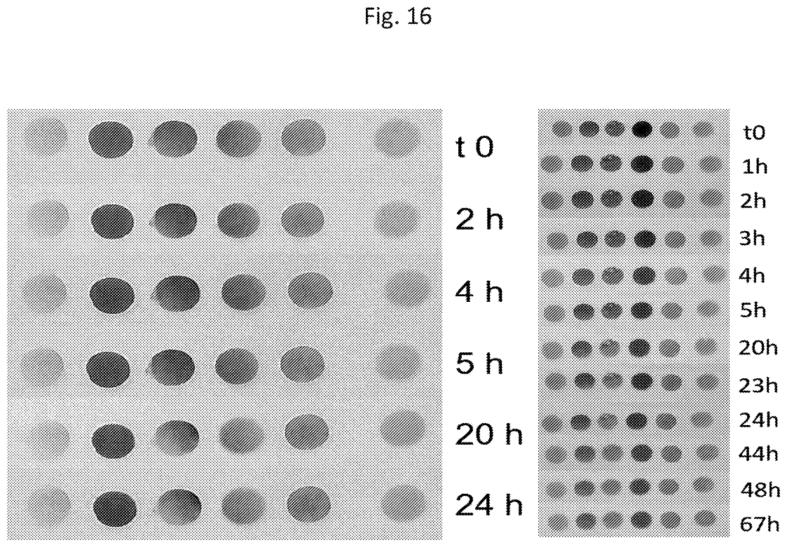

In FIG. 16, the comparison between the colors of the individual spots during the analysis in different samples is shown.

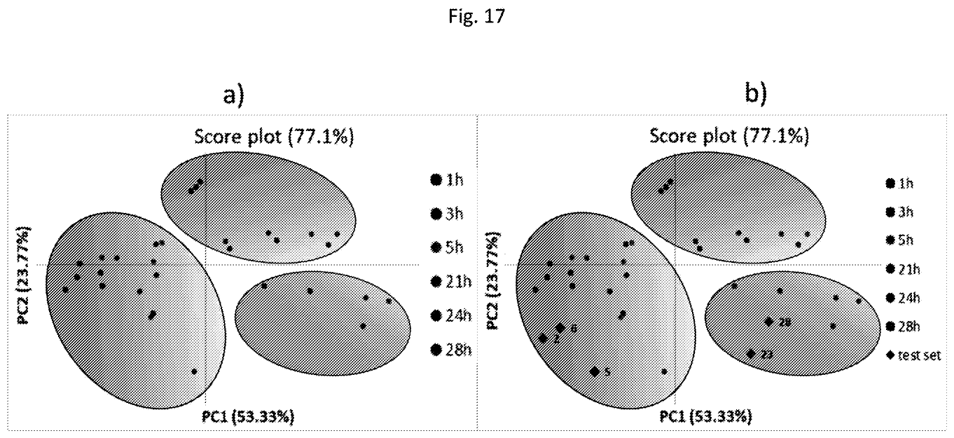

RGB codes of the individual spots of the array are sampled every time and the processing of those data is carried out with chemometric PCA, Principal Component Analysis, technique which allows to divide the degradation process in three main phases, called SAFE, WARNING and HAZARD, as shown in FIG. 17a.

The model thus obtained is validated by projecting within the unknown samples (FIG. 17b).

Finally, the most informative indicators for this food are selected, Bromothymol Blue (3), Thymol Blue (4) and Bromophenol Blue (5), and, for them, the reference colors related to the single degradation phases are collected.

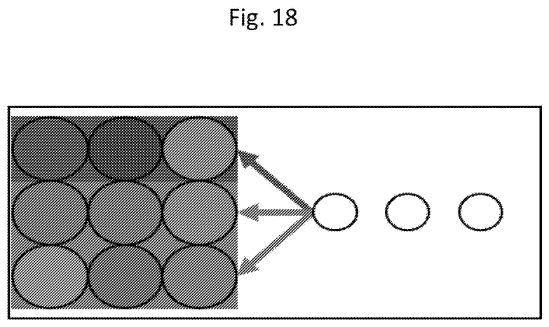

An example of possible label design, in which the sensors are placed in the white circles and may be directly compared with the legend printed on the left, is reported in FIG. 18.

The degradation monitoring is carried out at room temperature and in fridge. The instrumental analyses for the validation have not yet been carried out since the results above confirm the observed trend.

Monitoring on Fish Fillets

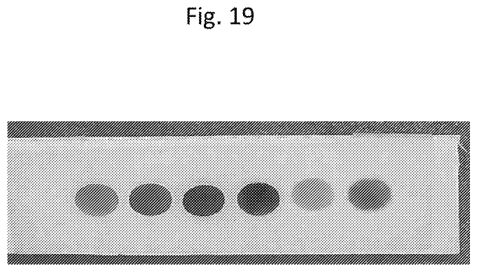

From the preliminary tests it is shown that, in the fish case, as observed with the device on ion exchanger, the first degradation phase is less extensive than observed in the chicken, while the second phase is earlier and more extensive. This difference is due to the lower presence of sugars in the fish, considered in fact a not-fat fresh food, and the abundance of nitrogen compounds that produce amines during degradation. For this reason, only the indicators from 1 to 4 are prepared in basic form and used for recognition of the acidic compounds characterizing the former degradation phase, through passage to acidic color; both indicator 5 and 6 instead are prepared in acidic form and pass to basic color when the production of acidic compounds ends. An example of the final array is shown in FIG. 19.

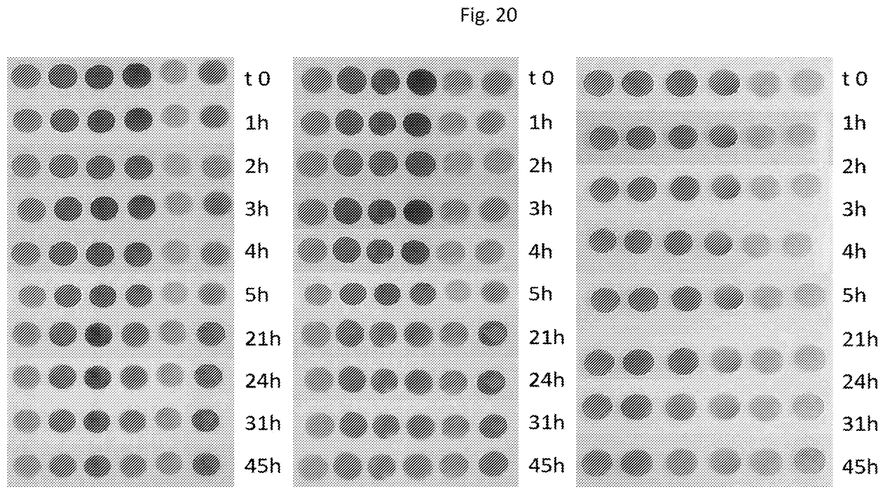

In FIG. 20, the comparison between the colors of the individual spots during the analysis in different samples is shown.

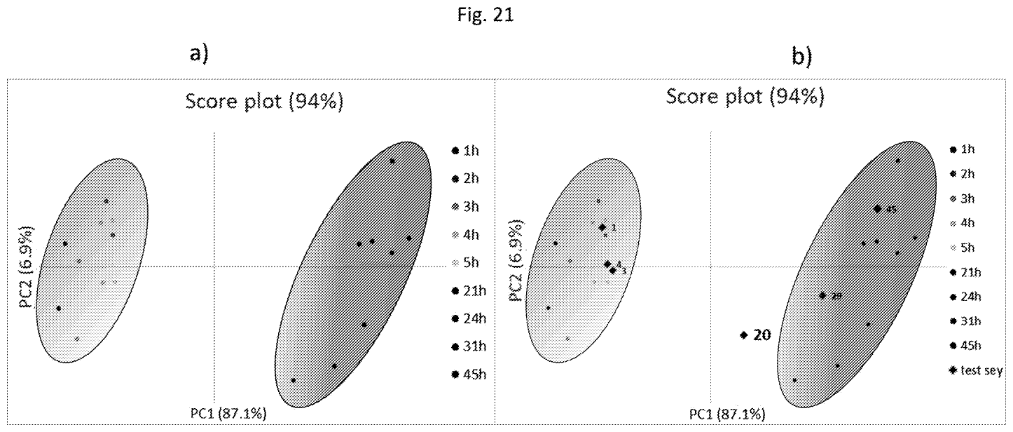

RGB codes of the individual spots of the array are sampled every time and the processing of those data is carried out with chemometric PCA, Principal Component Analysis, technique which allows to divide the degradation process in two main phases, called SAFE and HAZARD, as shown in FIG. 21a.

The model thus obtained is validated by projecting within the unknown samples (FIG. 21b), in such a way to verify the correct attribution, as well as with independent instrumental analyses.

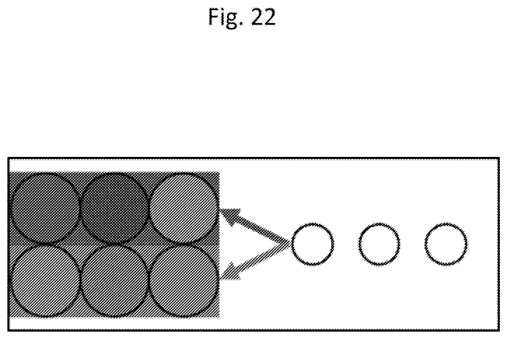

Finally, the most informative indicators for this food are selected, Bromothymol Blue (3), Thymol Blue (4) and Bromophenol Blue (5), and, for them, the reference colors related to the single degradation phases are collected. In FIG. 22, a possible label with the same design proposed for the chicken but with the correct legend in the case of fish is reported.

The degradation monitoring has been carried out, to date, at room temperature and in fridge. As for instrumental analyses instead, so far only some preliminary tests have been carried out which confirm the response of the sensor. However, more in-depth analyses are underway, similar to what has been done for the chicken meat.

Monitoring of Different Types of Milk

The sensor proposed has also been tested for the monitoring of samples of milk in order to extend the coverage as much as possible and demonstrate the versatility of the proposed device. For this type of fresh food, however, different modes from what was previously described have been used, because it is a liquid substance and for reasons related to the degradation process.

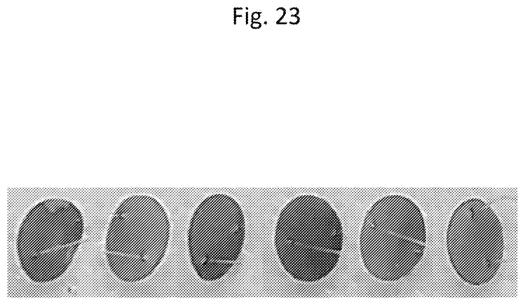

Firstly, the sensors are directly immersed in the sample and withdrawn at preset times for the photographs. As regards the more strictly scientific aspect instead, in the milk the two phases are not distinguished, but the degradation process consists in a consistent acidity increase in the fresh food up to exceeding the edibility threshold. For this reason, all sensors have been balanced to basic pH so as to verify which have a color change to a pH corresponding to the threshold of interest. An example of the final array on which the tests of viability were performed is shown in FIG. 23.

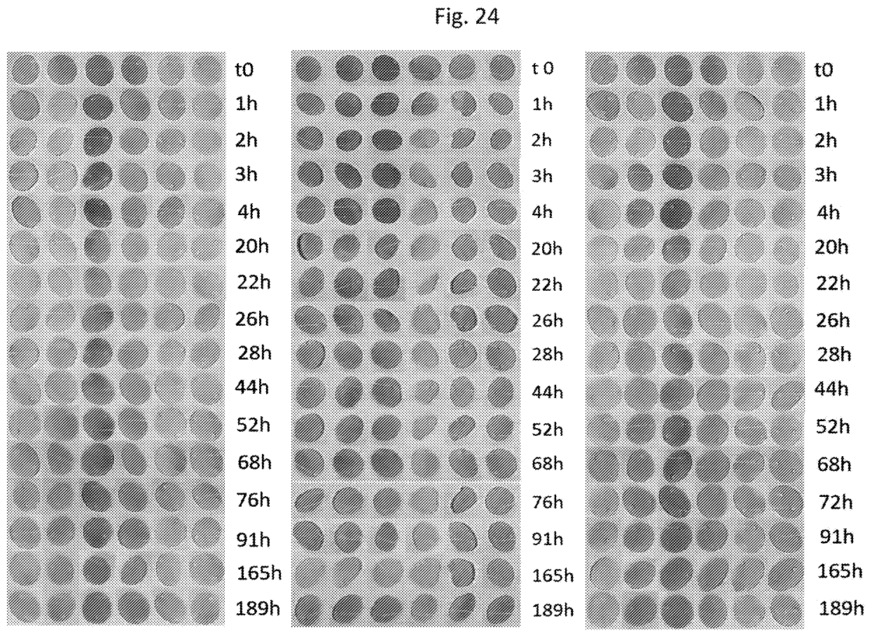

The analyses have been conducted on three types of milk, whole, semi-skimmed and skimmed, and at two different temperatures, room T and in fridge. In FIG. 24, the comparison between the sensor colors is reported for the three types of milk, preserved in fridge.

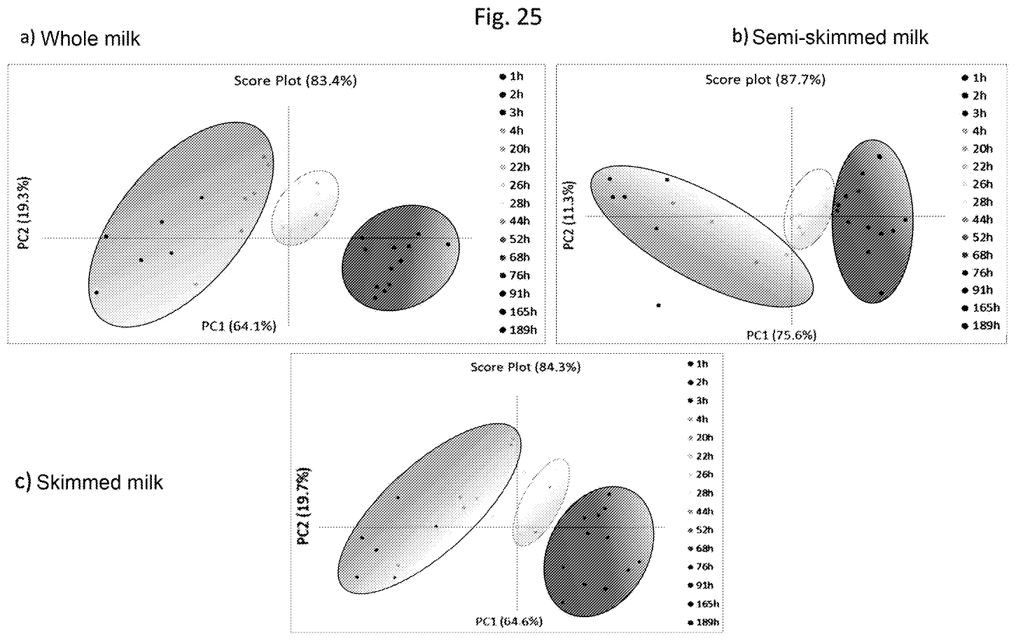

RGB codes of the individual spots of the array are sampled every time and the processing of those data is carried out with chemometric PCA, Principal Component Analysis, technique which allows for the three types divide the degradation process in three main phases, called SAFE, WARNING and HAZARD, as shown in FIG. 25 for the three types.

The validation procedure used is different: firstly, an acidic-basic titration is carried out to calculate the degree of acidity (ºSoxhlet-Henckel degrees, ºS.H.) and % w/V of lactic acid, two parameters commonly used for evaluating the goodness of milk, for the three types and in samples that the model classifies as SAFE and HAZARD. The findings of such determination, carried out each on three independent replicas, on milk samples pertaining to the three types: whole, semi-skimmed and skimmed, and standard deviations in parentheses.

| | ° S.H. | % w/V 1. a. | °S.H. | % w/V 1. a. |
|---|---|---|---|---|
| | SAFE | SAFE | HAZARD | HAZARD |
| Whole milk | 8.0 (1) | 180 (1) | 9.1 (1) | 204 (2) |
| Semi-skimmed | 7.9 (1) | 178 (2) | 8.7 (1) | 195 (1) |
| milk | | | | |
| Skimmed milk | 7.8 (1) | 176 (2) | 8.1 (1) | 183 (2) |

Note that the samples identified as HAZARD record higher values for both indicators that classify them as spoiled. Remember that normal milk must have values between 7 and 8, for ºS.H. and between 157 and 180 for % w/V in lactic acid. Instrumental analyses on the head space are also underway, with the purpose of determining volatile acids produced during degradation.

In this case, since what must be recorded is only the evolution towards lower pHs, only one sensor chosen on the base of results of the PCA is enough.

In this case, for this polymeric support the more sensitive sensor results to be Bromothymol Blue (3).

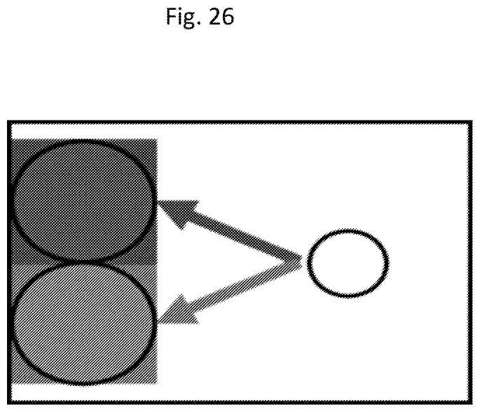

In FIG. 26, an example of possible label with the same design proposed for the chicken is reported, but with the sensor selected and the correct legend in the milk case.

### Example 5

### Possible Use of the System of Sensors as Litmus Paper

The signals are the RBG indexes, the response is the pH of the solutions, the partial regression of least squares (partial least square regression PLS) has been the tool used to shape the data set. The difficulty was to assign real pH values to the solutions contacted with the devices.

For the more acidic region, the pH assigned to the solution has been obtained from the mineral acid that has been directly titrated, for the neutral region, buffer solutions should be used. In this case, the true value has been assigned from measurements with the glass electrode.

The ultimate goal is to have the reference colors for a detection with the naked eye that allows to attribute the correct pH values to unknown samples.

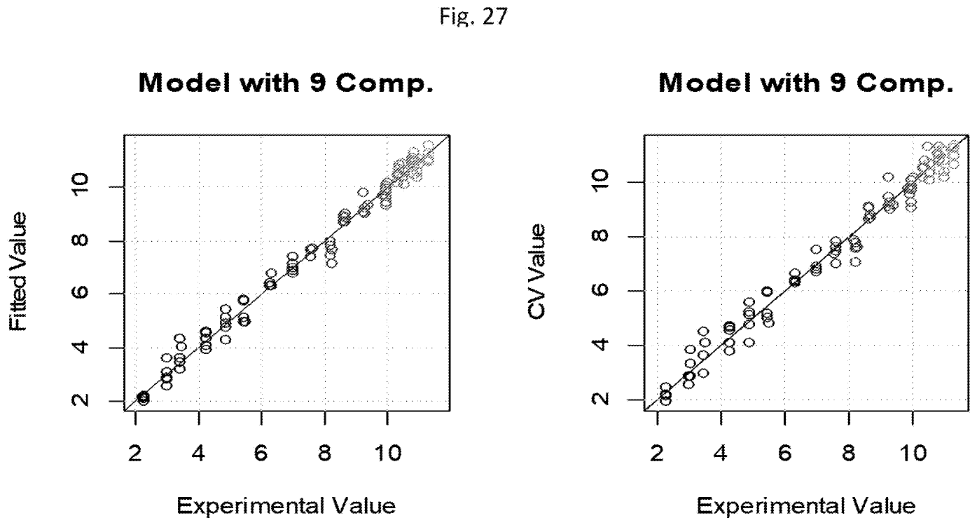

FIG. 27 shows the results of pH value modeling: experimental values with respect to calculated ones and the same in cross validation, as typical steps of PLS algorithm and compliance is definitely acceptable. In the Table below, the validation is shown of the model with external samples, and the pHs of the various solutions, not used for modeling, calculated by the colors are compared with those measured.

| BUFFER | I (M) | PHₑₓₚ | pH_{calc} | ΔpH |
|---|---|---|---|---|
| Acetate | 0.10 | 4.47 | 4.51 | 0.04 |
| PIPES | 0.10 | 7.3 | 6.99 | −0.31 |
| EPPS | 0.10 | 8.46 | 8.31 | −0.15 |
| Phthalate | 0.01 | 4.15 | 3.71 | −0.44 |
| phosphate | 0.01 | 7.61 | 7.20 | −0.41 |

From the above described, the benefits offered by present invention will be immediately apparent to the person skilled in the art.

First of all, the present invention provides a solution to the problem of not being able to identify the production of biogenic amines in the meat as a volatile compound, as, in real conditions and also in an advanced stage of decomposition, those compounds are not released except to a minimal extent, such to only minimally contribute to increase the pH.

Beneficially, it has been observed that the same system of sensors, that is, the same combination of two or three sensors, can be used for different types of meat; the person skilled in the art can indeed understand how the reference colors of safe, warning and hazard situations can be changed accordingly, or how the initial pH of the spots of indicators can be changed, to obtain the toning wanted, in light of the different composition in sugars, fats and proteins (and amino acidic composition of proteins), of the different types of meat.

What proposed by present invention allows to track the degradation process of fresh food over time.

It has also been noted that the method of the invention could be validly applied regardless of the preservation temperature of the meat; indeed, the course of degradation is the same, while the degradation timing is modified, obviously, slower if the meat is preserved at lower temperatures (as shown in FIG. 5B).

From the viewpoint of food companies, the system of the invention can be fully integrated in the product packaging process, without having to modify the chain of procedures.

The system of sensors is, moreover, immediate and easy to read for the consumer.

The system of sensors allows to check the freshness of the food without any analysis and without requiring the taking of a food sample.

Since the system also highlights the freshness of a product, it is possible to prevent wastes caused by the elimination of foodstuffs believed to be no longer eatable; indeed, the distribution entities (as, for example, the supermarkets) may have greater guarantees on the actual state of conservation of the product.

The use of the system of sensors of the invention as litmus paper has the advantage of being reversible, reliable, suitable not only for solutions but also for vapors, unlike the classic pH test papers.

What is claimed is:

1. A system of sensors sensitive to a feature indicative of freshness of a food represented by meat or of a food of animal origin, said system of sensors comprising a sensor comprising an indicator sensitive to pH change and sensor comprising a binder sensitive to thiols or sulfur-containing compounds concentration.

2. The system of sensors of claim 1, wherein said food is fresh.

3. The system of sensors of claim 1, further comprising an additional sensor sensitive to pH change.

4. The system of sensors of claim 3, wherein one sensor sensitive to pH change comprises an indicator that tones at acidic pH and is used in its acidic form or in its basic form.

5. The system of sensors of claim 3, wherein the additional sensor sensitive to pH change comprises an indicator that tones at neutral pH and is used in its acidic form or in its basic form.

6. The system of sensors of claim 1, wherein said sensor sensitive to pH change comprises an indicator selected from the group consisting of: 1-naphthoyl phthalein, thymolphthalein, phenolphthalein, phenol red, m-cresol purple, o-cresol red, bromothymol blue, thymol blue, T-Azo C (disodium salt of 2-[(1H-5-tetrazolyl)azo]-1,8-dihydroxynaphthalene-3,6-disulfonic acid trihydrate), Titan yellow, alizarin yellow, chlorophenol red, and bromophenol blue.

7. The system of sensors of claim 6, wherein said sensor sensitive to pH change comprises one or more indicators selected from the group consisting of: m-cresol purple, o-cresol red, bromothymol blue, thymol blue, chlorophenol red and bromophenol blue.

8. The system of sensors of claim 5, wherein said indicator is in its acidic form and is represented by chlorophenol red and bromophenol blue.

9. The system of sensors of claim 5, wherein said indicator is in its basic form and is represented by m-cresol purple, o-cresol red, bromothymol blue, thymol blue.

10. The system of sensors of claim 3, wherein the sensor sensitive to pH change comprises chlorophenol red as indicator, the additional sensor sensitive to pH change comprises bromothymol blue as indicator and the sensor sensitive to thiols or sulfur-containing compounds concentration comprises a binder represented by Ellman's reagent.

11. The system of sensors of claim 1, wherein said indicator and/or said binder are adsorbed or immobilized on a support.

12. The system of sensors of claim 11, wherein said support is represented by a polymeric material selected from the group consisting of: ethylene-vinyl-alcohol (EvOH), in particular, EvOH 27%, EvOH 29%, EvOH 32%, EvOH 38% or EvOH 44%, ethylene vinyl acetate (EVA), linear low density polyethylene modified with maleic anhydride (Orevac® 18362).

13. The system of sensors of claim 11, wherein said support is made of carboxymethyl cellulose.

14. The system of sensors of claim 11, wherein said support is a fabric or a non-woven fabric.

15. The system of sensors of claim 14, wherein said fabric and said non-woven fabric are of natural or synthetic fiber.

16. The system of sensors of claim 1, wherein said meat is chicken, beef, pork or fish meat.

17. The system of sensors of claim 11, wherein said support is represented by ethylene-vinyl-alcohol (EVOH) and said indicator is represented by: bromothymol blue, thymol blue, bromophenol blue.

18. The system of sensors of claim 1, wherein said meat is chicken or fish meat.

19. The system of sensors of claim 1, wherein said food of animal origin is milk.

20. The system of sensors of claim 1, wherein said indicator is represented by bromothymol blue.

21. A process for preparing a support of a system of sensors sensitive to a feature indicative of freshness of a food represented by meat or of a food of animal origin, said system of sensors comprising a sensor comprising an indicator sensitive to pH change and a sensor comprising a binder sensitive to thiols or sulfur-containing compounds concentration, wherein said support is represented by carboxymethyl cellulose or by a polymeric material selected from the group consisting of: ethylene-vinyl-alcohol (EvOH), in particular, EvOH 27%, EvOH 29%, EvOH 32%, EvOH 38% or EvOH 44%, ethylene vinyl acetate (EVA), linear low density polyethylene modified with maleic anhydride (Orevac® 18362), said process comprising the steps of:
1) functionalizing said indicator or said binder, and
2) derivatizing said polymeric material or carboxymethyl cellulose with the indicator or the functionalized binder obtained from step 1).

22. A process for preparing a support of a system of sensors sensitive to a feature indicative of freshness of a food represented by meat or of a food of animal origin, said system of sensors comprising a sensor comprising an indicator sensitive to pH change and a sensor comprising a binder sensitive to thiols or sulfur-containing compounds concentration, wherein said support is a fabric or a non-woven fabric, said process comprising adsorbing said indicator or said binder on said fabric or non-woven fabric.

23. A container or a container element for a fresh food represented by meat or a food of animal origin or milk, comprising the system of sensors of claim 1.

24. The system of sensors of claim 12 as reversible pH indicator.

25. The system of sensors of claim 24 as litmus paper.
